(19) United States
(12) Patent Application Publication
(10) Pub. No.: US 2024/0202623 A1
(43) Pub. Date: Jun. 20, 2024

(54) HUMAN DIGITAL TWINNING METHOD AND SYSTEM OF EMOTIONAL REGULATION FOR EMOTIONAL LABOR IN WORKPLACES USING MULTI-MODAL SENSOR DATA

(71) Applicant: Korea Advanced Institute of Science and Technology, Daejeon (KR)

(72) Inventors: Uichin LEE, Daejeon (KR); Duri LEE, Daejeon (KR); Eunji PARK, Daejeon (KR); Young Ji KOH, Daejeon (KR); Yunjo HAHN, Daejeon (KR)

(21) Appl. No.: 18/212,435

(22) Filed: Jun. 21, 2023

(30) Foreign Application Priority Data
Dec. 14, 2022 (KR) 10-2022-0174693
Mar. 23, 2023 (KR) 10-2023-0038174

(51) Int. Cl. G06Q 10/0631; A61B 5/16

(52) U.S. Cl. CPC G06Q 10/063114; A61B 5/165

(57) ABSTRACT

Disclosed is a method and system for implementing a human digital twin of an emotional worker related to emotional regulation that results from a job using multimodal sensor data. A data collection method for implementing a human digital twin performed by a data collection system may include collecting multimodal data through heterogeneous data collection tools; and determining emotional regulation information that results from an emotional worker through an emotional labor model using the collected multimodal data.

### CROSS-REFERENCE TO RELATED APPLICATIONS

This application claims the priority benefit of Korean Patent Applications No. 10-2022-0174693, filed on Dec. 14, 2022 and No. 10-2023-0038174, filed on Mar. 23, 2023, in the Korean Intellectual Property Office, the disclosure of which is incorporated herein by reference.

### BACKGROUND

### 1. Field of the Invention

Example embodiments relate to technology for determining emotional regulation of an emotional worker.

### 2. Description of the Related Art

According to an increase in the demand for mental health care in the market through the 4-th industrial revolution and the COVID-19 pandemic, digital healthcare services surged. In parallel with this, there have been continuous reports related to mental health and physical health caused by the stress of people engaged in occupations represented by emotional workers. Each country has prepared institutional measures, for example, legislation and medical and service support, to protect people engaged in emotional labor from excessive stress. Studies to identify the mechanism of “emotional labor” in which workers in emotional labor occupations perform services are continuously being conducted in the field of psychology. Emotional labor refers to the effort that a worker internally puts into performing a required behavior (behavior that needs to be expressed) different from behavior that the worker actually perceives. The emotional worker performs such internal effort by unconsciously selecting two strategies, surface acting (SA) and deep acting (DA), and in this process, psychological labor acting is performed. The surface acting (SA) relates to simply expressing a required emotion regardless of the emotion the corresponding worker perceives and deep acting relates to expressing an emotion as a result of trying to assimilate an actual emotion into the required emotion.

In parallel with social change, advancements in sensor, big data, and artificial intelligence (AI) technology are continuously made in terms of science and technology. Research is being conducted to data, diagnose, and manage internal response factors of a human, such as stress, using a multimodal sensor. In particular, representative research that measures affective using multimodal sensor data includes the WESAD dataset study 2017.

Closely to this, the concept of human digital twin that applies, to humans, digital twin technology for performing diagnosis and prediction by simulating the real world in a virtual space is being researched. The diagnosis and the prediction are performed through real-time simulation in the virtual space. In particular, studies are being conducted to implement a mental digital twin for mental health management, which is difficult to identify externally clearly but is likely to cause quite fatal results (due to its gradual nature).

Various physical health, mental health, and behavioral effects occur as a functioning result of external causes that constitute the mechanisms of job stress and emotional regulation and individual intrinsic factors and mechanism vary from person to person. Various data may be collected, evaluated, and predicted in real-time using a sensor. However, it is essential to consider such data characteristics when implementing a human digital twin for emotional workers that provide personalized services for job stress and emotional regulation in a large-scale system.

### SUMMARY

Example embodiments may provide a system and method for measuring a level of emotional regulation that results from a job of an emotional worker using multimodal data.

Example embodiments may provide a system and method that may measure an emotional regulation level using an emotional labor model, organically manipulate various external interfaces to utilize information related to the measured emotional regulation, and be optimized for individuals.

Example embodiments may be used as a representative system architecture for implementing a human digital twin that is predicted to potentially interconnect various heterogeneous equipment and systems.

Example embodiments may apply multimodal data collected through heterogeneous data collection tools to the implementation of a digital twin system of emotional workers engaged in emotional labor occupations (e.g., call center, service position, teacher, etc.).

According to an aspect of at least one example embodiment, there is provided a data collection method for implementing a human digital twin performed by a data collection system, the data collection method includes collecting multimodal data through heterogeneous data collection tools; and determining emotional regulation information that results from a job of an emotional worker through an emotional labor model using the collected multimodal data.

The collecting may include classifying emotional workers according to a pre-defined digital twin classification model from the collected multimodal data.

The collecting may include classifying necessary data from the collected multimodal data according to specifications that have contain a type of multimodal data or a data collection period defined in a digital twin classification model of the emotional worker.

The collecting may include preprocessing for inputting the classified necessary data into the emotional labor model.

The heterogeneous data collection tools may include an aperiodic synchronization data collection tool or a periodic synchronization data collection tool. The aperiodic synchronization data collection tool may aperiodically collect personal information or self-reported information input from the emotional worker, and the periodic synchronization data collection tool may periodically collect biometric data, sensor data, voice data, and environmental information.

The data collection method may further include performing user authentication of the emotional worker in response to an access request for collecting the multimodal data, granting a right to access the multimodal data collected about to with concerning the emotional worker through the performed user authentication, and storing the multimodal data to which the right to access is assigned.

The storing may include storing self-reported information and personal information that are input from the emotional worker and the emotional regulation information that results from the job of the emotional worker determined through the emotional labor model.

The determining may include quantifying the emotional regulation information of the emotional worker through the emotional labor model using heterogeneous multimodal data and updating the emotional labor model through the feedback of the emotional worker on the quantified emotional regulation information.

According to an aspect of at least one example embodiment, there is provided a non-transitory computer-readable recording medium storing instructions that, when executed by a processor, cause the processor to implement a data collection method for implementing a human digital twin performed by a data collection system, the data collection method including collecting multimodal data through heterogeneous data collection tools; and determining emotional regulation information that results from a job of an emotional worker through an emotional labor model using the collected multimodal data.

According to an aspect of at least one example embodiment, there is provided a data collection system including a data collector configured to collect multimodal data through heterogeneous data collection tools; and an emotional regulation determiner configured to determine emotional regulation information that results from a job of an emotional worker through an emotional labor model using the collected multimodal data.

According to some example embodiments, collecting data necessary for a system to be implemented without separately manipulating a sensor collection environment around a user, is possible collect heterogeneous multimodal data.

Psychological mechanisms, such as emotional labor, emotional regulation, and the like, may use various variables as data, may be measured using various types of sensors, and may efficiently process data for implementation of a platform that integrates a large amount of data through a process of selecting data optimized for individuals.

Further areas of applicability will become apparent from the description provided herein. The description and specific examples in this summary are intended for illustration only and not to limit the scope of the present disclosure.

### BRIEF DESCRIPTION OF THE DRAWINGS

These and/or other aspects, features, and advantages of the invention will become apparent and more readily appreciated from the following description of embodiments, taken in conjunction with the accompanying drawings of which:

FIG. 1 is a diagram illustrating a configuration of a data collection system according to an example embodiment;

FIG. 2 is a flowchart illustrating a data collection method for implementing a human digital twin according to an example embodiment;

FIG. 3 is a flowchart illustrating a data collection operation between components of a data collection system according to an example embodiment; and

FIG. 4 is a flowchart illustrating a data collection operation according to an example embodiment.

### DETAILED DESCRIPTION

Hereinafter, example embodiments will be described with reference to the accompanying drawings.

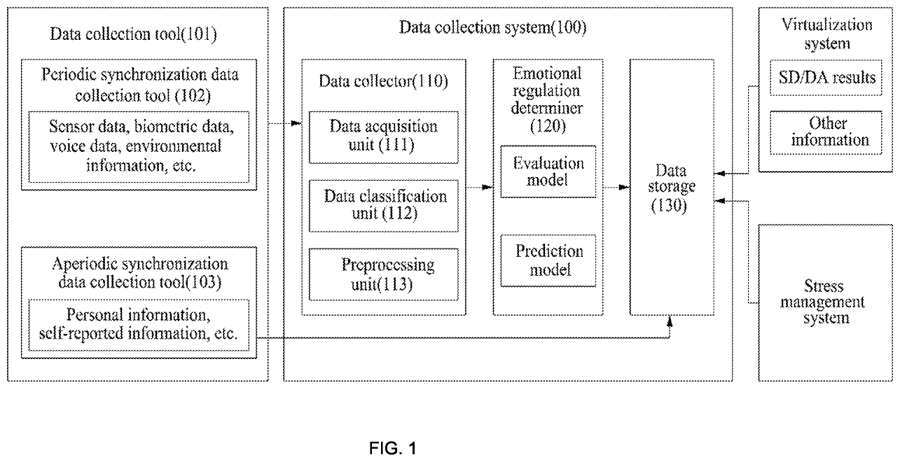

FIG. 1 is a diagram illustrating a configuration of a data collection system according to an example embodiment.

A data collection system 100 may continuously update and collect a type of data and a data collection period according to data specifications optimized for emotional regulation prediction through a personalized classification model, using multimodal data that is collected by heterogeneous data collection tools.

The data collection system 100 relates to collecting multimodal data to implement a human digital twin and may include a data collector 110, an emotional regulation determiner 120, and data storage 130.

The data collection system 100 may be partially hardware and partially software in some aspects. Components of the data collection system 100 may be implemented through devices for transmitting and receiving data of a specific format and contents using electronic communication and software for supporting operations. For example, a data acquisition unit 111, a data classification unit 112, and a preprocessing unit 113 of the data collector 110 may be software implemented through an application of an electronic device and the emotional regulation determiner 120 and the data storage 130 may be implemented through a single device having data storage and processing capability, such as a large capacity server. Also, heterogeneous data collection tools, a virtualization system, and an external stress management system are not illustrated in the contents of the present invention but are illustrated to help to understand since they are required to understand the core of the present invention and elements that implement the human digital twin as a whole.

The data collector 110 may collect multimodal data through heterogeneous data collection tools. The heterogeneous data collection tools may measure and receive multimodal data through a service that is executed by hardware or software, such as the electronic device. They may include a periodic synchronization data collection tool 102 or an aperiodic synchronization data collection tool 103. For example, the periodic synchronization data collection tool 102 may acquire biometric data, sensor data, voice data, and environmental information periodically (e.g., every preset time). The periodic synchronization data collection tool 102 may acquire the biometric data through a sensor that is provided to a wearable device (e.g., smartwatch), may collect the voice data through a microphone, audio, or a recorder that is provided to the wearable device or a voice collection tool, and may collect the environmental information, such as weather, humidity, and temperature, provided from an environmental information collection tool or an outside. In addition, the periodic synchronization data collection tool 102 may collect data through other various collection tools. The aperiodic synchronization data collection tool 103 may aperiodically collect personal information or self-reported information input from an emotional worker through a data collection service for implementing the human digital twin.

The data collector 110 serves to select and collect optimized data to evaluate an emotional regulation level of the user and may include the data acquisition unit 111, the data classification unit 112, and the preprocessing unit 113.

The data acquisition unit 111 may acquire the collected multimodal data from a data collection tool 101 that includes the periodic synchronization data collection tool 102 or the aperiodic synchronization data collection tool 103.

The data classification unit 112 may classify emotional workers using a pre-defined digital twin classification model from the collected multimodal data. For example, the data classification unit 112 may receive personal characteristic information in a self-report form and classify emotional workers according to a plurality of (n) digital twin classification models related to previously defined emotional workers. For example, although counseling work is performed in the same contact center, a model may vary depending on whether the counseling work is telephone counseling, face-to-face counseling, or chat counseling. Also, even in the same telephone counseling occupational group, keyboard usage differs according to the characteristic of counseling contents and the type of client counseling inquiry. That is, due to a difference in the type of information received from sensor data, a composition of a model may vary. Also, a model to be applied may vary depending on demographic information (e.g., age and gender) known to have a close causal relationship with job stress and individual personality factors. In some example embodiments, a model in which results of self-questioning and answering results about a current situation of an individual (e.g., stress level and personal grievance) aperiodically received from an emotional worker are applied may be applied. The data classification unit 112 may continuously update newly developed models. Here, a digital twin classification model used as a standard is a model that is pre-defined through empirical research, and a type of optimized multimodal data and a data collection period are defined for each digital twin classification model.

The data classification unit 112 may classify necessary data from the collected multimodal data according to specifications that include a type of sensor data or the data collection period defined in the digital twin classification model of the emotional worker. Necessary information may vary depending on a digital twin classification model, and many or few options may be collected according to a type of sensor used. For example, location information (e.g., global positioning system (GPS), etc.) is not acquired for a user that statically performs emotional labor in a call center, and location information (e.g., GPS, etc.) needs to be acquired for a user that physically performs emotional labor in the field.

The preprocessing unit 113 may perform preprocessing to input the classified essential data to an emotional labor model. The preprocessing unit 113 may modify the classified necessary data to be optimized for the emotional labor model. For example, when heart rate (HR) data is included in the classified necessary data, the preprocessing unit 113 may process the HR data into information, such as mean and STD, according to a required period. For example, when voice data is included in the classified required data, the preprocessing unit 113 may extract voice information that provides for a format of voice data and a length of voice data required by the emotional labor model. In detail, since a telephone counseling-oriented worker as a type of emotional laborer is required to have a constant and kind change in voice tone or speed, the preprocessing unit 113 may extract the frequency of content, such as the number of times a specific expression, “Sorry” or “That is difficult,” repeats through preprocessing of the voice data, rather than extracting pitch or speed information. Even in the case of the same voice data, if an emotional worker works for socially underprivileged (e.g., children, the elderly, the disabled) that cannot resist, the preprocessing unit 113 may select and preprocess frequency, loudness, and intensity that are phonetic characteristics, as necessary data.

The emotional regulation determiner 120 may determine emotional regulation information resulting from the job of the emotional worker through the emotional labor model using the collected multimodal data. Here, emotional regulation relates to explaining an individual's time and emotion, a method of experiencing and expressing an emotion, and a process and the effect thereof. Here, the emotional labor model may be trained to determine the emotional regulation resulting from the job of the emotional worker using a dataset related to the job of the emotional worker. The emotional regulation determiner 120 may quantify an emotional labor state of a user in real-time using an evaluation model and a prediction model for the collected multimodal data. For example, the evaluation model and the prediction model may be updated with the development of a new algorithm using various machine learning algorithms that include a general linear model. Also, the evaluation model may verify accuracy by periodically requesting user feedback, and the prediction model may calculate the accuracy with feedback results on how accurate evaluation model results are derived based on the prediction model. The evaluation model and the prediction model may be continuously updated based on user feedback.

The data storage 130 may store self-reported information and personal information that are input from the emotional worker and the emotional regulation information that results from the job of the emotional worker determined through the emotional labor model. The data storage 130 may store aperiodically synchronized data, such as personal information, self-reported information, and feedback information on prediction model results. The data storage 130 may store data acquired through the data collector 110.

The data collection system 100 may exchange data with an external virtualization system through the data storage 130. For example, the data collection system 100 may store multimodal data collected through the aperiodic synchronization data collection tool 103, evaluation of an emotional regulation level determined by the emotional labor model, and prediction result data in the data storage 130. The external virtualization system may request and virtualize necessary information according to a type of a user interface (e.g., mobile, tablet, large monitor, etc.). The virtualization system does not require separately storing data and may use data of the data storage 130 depending on a type of device to be implemented and the purpose of the system. The data storage 130 may collect data object information (emotional worker) or information on an interface type frequently requested by the virtualization system and may use the collected information as additional data.

Also, the data storage 130 may exchange data with a newly developed stress management system. For example, a performance level of emotional labor known to be closely related to stress assist in analyzing various stress management systems. Also, an individual stress level may be used to predict a future performance level of emotional labor.

Also, the data storage 130 may guarantee data security using an authentication procedure when accessing and data encryption. Here, detailed specifications regarding a storage form of data stored in the data storage 130 and an interworking method may be modified according to a system configuration.

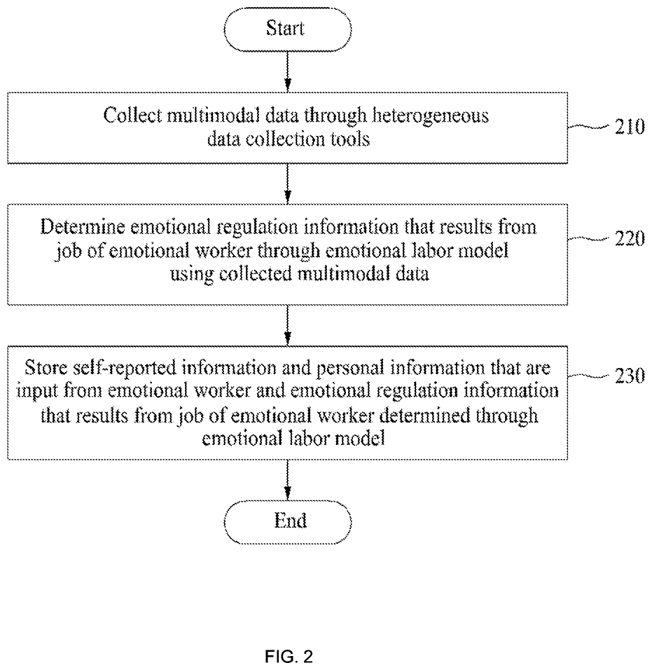

FIG. 2 is a flowchart illustrating a data collection method for implementing a human digital twin according to an example embodiment.

Referring to FIG. 2, in operation 210, a data collection system may collect multimodal data through heterogeneous data collection tools. The data collection system may organize emotional workers according to a pre-defined digital twin classification model from the collected multimodal data. The data collection system may classify necessary data from the collected multimodal data according to specifications that include a type of multimodal data or a data collection period defined in a digital twin classification model of the emotional worker. The data collection system may perform preprocessing to input the classified necessary data into the emotional labor model.

In operation 220, the data collection system may determine emotional regulation information that results from the job of the emotional worker through the emotional labor model using the collected multimodal data. For example, the data collection system may quantify the emotional regulation information as a numerical value between 0 and 100. Also, the emotional labor model may determine the emotional regulation information by receiving at least one biometric data, sensor data, voice data, environmental information, personal information, and self-reported information among the collected multimodal data. Alternatively, the emotional labor model may determine the emotional regulation information using data collected from a periodic synchronization data collection tools and an aperiodic synchronization data collection tool among the collected multimodal data. The data collection system may quantify the emotional regulation information of the emotional worker in real time through the emotional labor model using heterogeneous multimodal data. It may update the emotional labor model through the feedback from the emotional worker for the quantified emotional regulation information.

In operation 230, the data collection system may store self-reported information and personal information that are input from the emotional worker and emotional regulation information that results from the job of the emotional worker determined through the emotional labor model. The data collection system may perform user authentication of the emotional worker in response to an access request for collecting multimodal data, may grant a right to access the multimodal data collected about the emotional worker through the performed user authentication, and may store the multimodal data to which the right to access is granted.

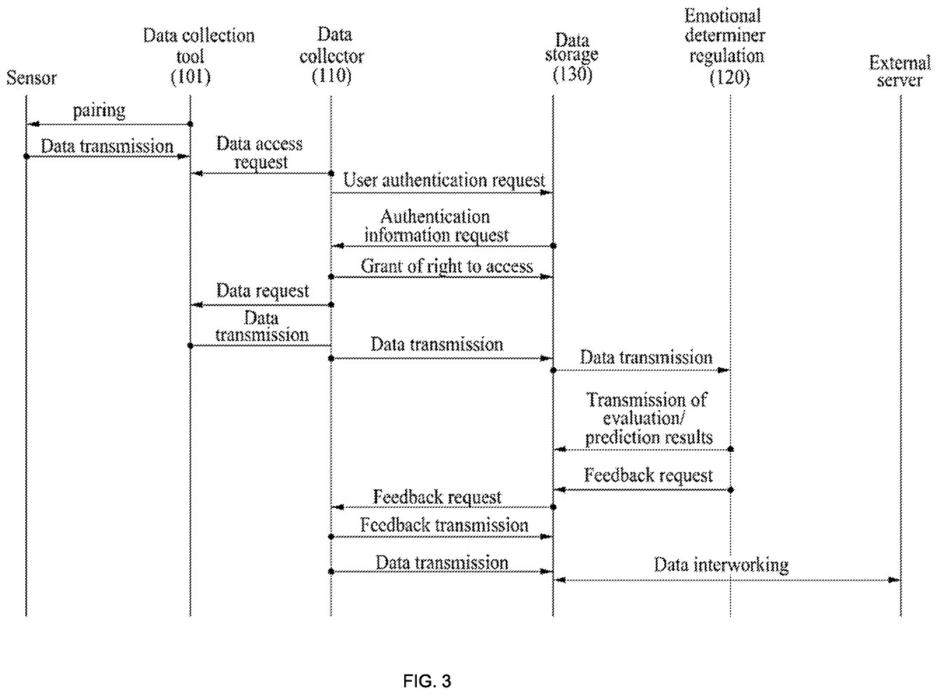

FIG. 3 is a flowchart illustrating a data collection operation between components of a data collection system according to an example embodiment.

An operation of collecting data through an operation with a sensor, the data collection tool 101, the data collector 110, the emotional regulation determiner 120, the data storage 130, and an external server is described with reference to FIG. 3.

The sensor may measure sensor data. The data collection tool 101 may be paired with the sensor and may receive the measured sensor data that is delivered from the sensor. The data collector 110 may request access to the data received by the data collection tool 101. The data collector 110 may request user authentication from the data storage 130. The data storage 130 may request authentication information from the data collector 110. The data collector 110 may authentically authenticate the emotional worker and may grant a right to access sensor data collected in relation to the emotional worker through the performed user authentication. The data collector 110 may request data from the data collection tool 101. The sensor data received by the data collection tool 101 may be transmitted to the data collector 110. The data collector 110 may share the sensor data from the data collection tool 101 to the data storage 130. The data storage 130 may transmit the received sensor data to the emotional regulation determiner 120. The emotional regulation determiner 120 may acquire evaluation and prediction results of emotional regulation information using the received sensor data and transmit the acquired evaluation and prediction result data to the data storage 130. Also, the emotional regulation determiner 120 may request feedback on the evaluation and prediction result data from the data storage 130. The data storage 130 may request feedback from the data collector 110. The data collector 110 may transmit feedback information on a feedback request to the data storage 130. A session between the data collector 110 and the data storage 130 may be terminated. Data stored in data storage 130 may be shared with the external server through data interworking between data storage 130 and the external server.

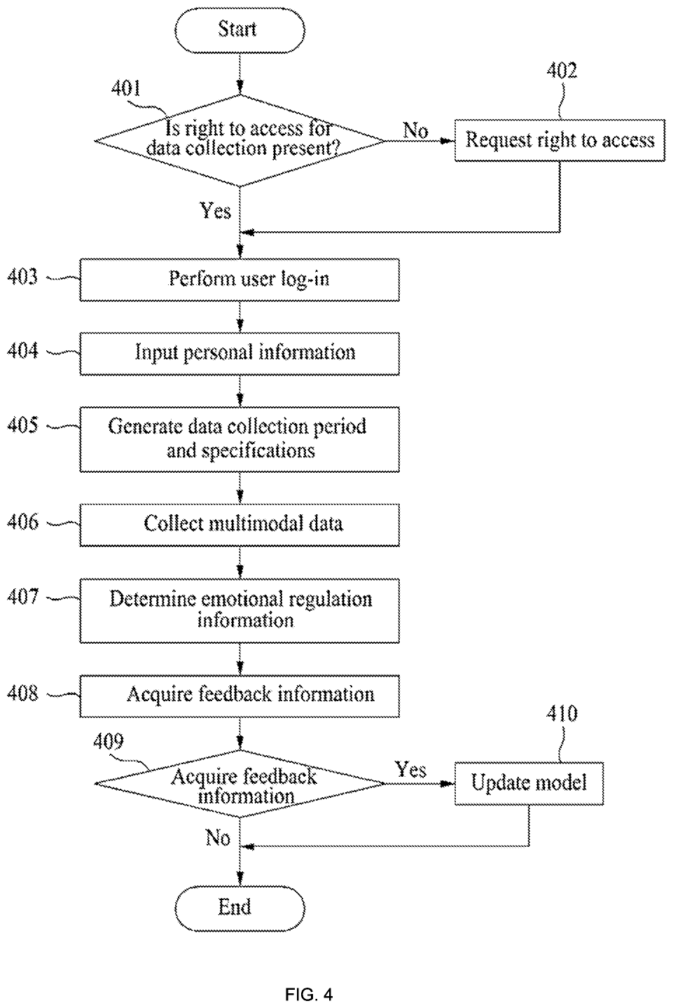

FIG. 4 is a flowchart illustrating a data collection operation using an example embodiment.

Referring to FIG. 4, in operation 401, the data collection system may determine whether a right to access data collection is present. In operation 402, when the right to access data collection is absent, the data collection system may request the right to access. In operation 403, log-in may be performed so that a user enters an ID and a password into a data collection service for implementing a human digital twin. In operation 404, as the user logs in, the data collection system may receive input of personal information. In operation 405, the data collection system may generate data collection period and specifications. Here, the data collection period and the specifications may be preset. Also, the data collection period and the specifications may be generated according to the settings of the user or an administrator. In operation 406, the data collection system may collect multimodal data. In operation 407, the data collection system may determine emotional regulation information through the emotional labor model using multimodal data. In operation 408, the data collection system may acquire feedback information input from the user. For example, the user may view determination result data and prediction result data derived through the emotional labor model generate feedback related to an accuracy/inaccuracy status, and transmit the generated feedback information to the data collection system. In operation 409, the data collection system may determine whether changing the emotional labor model is necessary based on the acquired feedback information. When it is determined necessary to change the emotional labor model, the data collection system may update the emotional labor model in operation 410. When it is determined that changing the emotional labor model is unnecessary, the data collection system may terminate the process.

The systems and/or apparatuses described herein may be implemented using hardware components, software components, and/or a combination thereof. For example, apparatuses and components described herein may be implemented using one or more general-purpose or special-purpose computers, such as, for example, a processor, a controller, an arithmetic logic unit (ALU), a digital signal processor, a microcomputer, a field programmable gate array (FPGA), a programmable logic unit (PLU), a microprocessor, or any other device capable of responding to and executing instructions in a defined manner. A processing device may run an operating system (OS) and one or more software applications that run on the OS. The processing device also may access, store, manipulate, process, and create data in response to the execution of the software. For the purpose of simplicity, the description of a processing device is used as singular; however, one skilled in the art will appreciate that the processing device may include multiple processing elements and/or multiple types of processing elements. For example, the processing device may include multiple processors or a processor and a controller. In addition, different processing configurations are possible, such as parallel processors.

The software may include a computer program, a piece of code, an instruction, or some combinations thereof, for independently or collectively instructing or configuring the processing device to operate as desired. Software and/or data may be embodied in any type of machine, component, physical equipment, virtual equipment, computer storage medium or device or in a propagated signal wave capable of providing instructions or data to or being interpreted by the processing device. The software also may be distributed over network—coupled computer systems so that the software is stored and executed in a distributed fashion. The software and data may be stored by one or more computer-readable storage mediums.

The methods, according to the example embodiments, may be recorded in non-transitory computer-readable media, including program instructions to implement various operations embodied by a computer. Also, the media may include, alone or in combination with program instructions, data files, data structures, and the like. Program instructions stored in the media may be specially designed and constructed for the example embodiments, or they may be well-known and available to those with computer software arts skills. Examples of non-transitory computer-readable media include magnetic media such as hard disks, floppy disks, and magnetic tapes; optical media such as CD ROM disks and DVDs; magneto-optical media such as floptical disks; and hardware devices that are specially to store and perform program instructions, such as read-only memory (ROM), random access memory (RAM), flash memory, and the like. Examples of program instructions include machine code, such as produced by a compiler, and files containing higher-level code that may be executed by the computer using an interpreter.

While this disclosure includes specific example embodiments, it will be apparent to one of ordinary skill in the art that various alterations and modifications in form and details may be made in these example embodiments without departing from the spirit and scope of the claims and their equivalents. For example, suitable results may be achieved if the described techniques are performed in a different order, and/or if components in a described system, architecture, device, or circuit are combined in a different manner, and/or replaced or supplemented by other components or their equivalents. Therefore, the scope of the disclosure is defined not by the detailed description, but by the claims and their equivalents, and all variations within the scope of the claims and their equivalents are to be construed as being included in the disclosure.

What is claimed is:

1. A data collection method for implementing a human digital twin performed by a data collection system, the data collection method comprising
collecting multimodal data through heterogeneous data collection tools; and
by using the collected multimodal data to determine emotional regulation information that results from a job of an emotional worker through an emotional labor model.

2. The data collection method of claim 1, wherein the collecting comprises classifying emotional workers according to a pre-defined digital twin classification model from the collected multimodal data.

3. The data collection method of claim 1, wherein the collecting comprises classifying necessary data from the collected multimodal data according to specifications that include a type of multimodal data or a data collection period defined in a digital twin classification model of the emotional worker.

4. The data collection method of claim 3, wherein the collecting comprises performing preprocessing for inputting the classified necessary data to the emotional labor model.

5. The data collection method of claim 1, wherein the heterogeneous data collection tools include an aperiodic synchronization data collection tool or a periodic synchronization data collection tool,
the aperiodic synchronization data collection tool aperiodically collects personal information or self-reported information input from the emotional worker, and the periodic synchronization data collection tool periodically collects biometric data, sensor data, voice data, and environmental information.

6. The data collection method of claim 1, further comprising:
performing user authentication of the emotional worker in response to an access request for collecting the multimodal data, granting a right to access the multimodal data collected about to with concerning the emotional worker through the performed user authentication, and storing the multimodal data to which the right to access is assigned.

7. The data collection method of claim 6, wherein the storing comprises storing self-reported information and personal information that is input from the emotional worker, and the emotional regulation information that results from the job of the emotional worker determined through the emotional labor model.

8. The data collection method of claim 1, wherein the determining comprises:
quantifying the emotional regulation information of the emotional worker through the emotional labor model using heterogeneous multimodal data and updating the emotional labor model through the feedback of the emotional worker on the quantified emotional regulation information.

9. A non-transitory computer-readable recording medium storing instructions that, when executed by a processor, cause the processor to implement a data collection method for implementing a human digital twin performed by a data collection system, the data collection method comprising:
collecting multimodal data through heterogeneous data collection tools; and
by using the collected multimodal data to determine emotional regulation information that results from a job of an emotional worker through an emotional labor model.

10. A data collection system comprising:
a data collector configured to collect multimodal data through heterogeneous data collection tools; and
an emotional regulation determiner configured to determine emotional regulation information that results from a job of an emotional worker through an emotional labor model using the collected multimodal data.
